(19) United States
(12) Patent Application Publication
(10) Pub. No.: US 2024/0330657 A1
(43) Pub. Date: Oct. 3, 2024

(54) TRAINING METHOD AND DEVICE FOR MOLECULAR GENERATION MODEL

(71) Applicant: UIF (University Industry Foundation), Yonsei University, Seoul (KR)

(72) Inventors: Sanghyun PARK, Seoul (KR); Jonghwan CHOI, Incheon (KR); Sangmin SEO, Seoul (KR)

(73) Assignee: UIF (University Industry Foundation), Yonsei University, Seoul (KR)

(21) Appl. No.: 18/399,450

(22) Filed: Dec. 28, 2023

(30) Foreign Application Priority Data
Apr. 3, 2023 (KR) 10-2023-0043303

(51) Int. Cl. G06N 3/0455; G16B 15/30; G16B 40/20; G16C 20/50

(52) U.S. Cl. CPC G06N 3/0455; G16B 15/30; G16B 40/20; G16C 20/50

(57) ABSTRACT

Disclosed is a method for a molecular generation model including obtaining a dataset including a source molecular model, a target molecular model whose structural similarity with the source molecular model exceeds a first threshold, and a negative molecular model whose structural similarity with one or more models of the source molecular model or the target molecular model is smaller than or equal to the first threshold, training the molecular generation model to adjust a distance between the source molecular model and the target molecular model based on the dataset and a first loss function, and training the molecular generation model to adjust at least one distance of a distance between the source molecular model and the negative molecular model, and a distance between the target molecular model and the negative molecular model based on the dataset and a second loss function different from the first loss function.

### CROSS-REFERENCE TO RELATED APPLICATIONS

This application claims priority under 35 U.S.C. § 119 to Korean Patent Application No. 10-2023-0043303 filed on Apr. 3, 2023, in the Korean Intellectual Property Office, the disclosures of which are incorporated by reference herein in their entireties.

### TECHNICAL FIELD

Embodiments of the present disclosure described herein relate to a training method and device for molecular generation model.

### BACKGROUND

Structure-constrained molecule generation is a challenging problem in goal-directed molecular optimization research. The structure-constrained molecule generation aims to generate new molecules that are similar to the molecular structure of the source drug but with improved target chemical properties. The conventional approach in organic chemistry includes investigating a main region, which binds to a target biological entity, in the structure of the source drug to identify new drug candidates with potential activity against specific diseases, and identifying the molecular structure in consideration of the combination of molecular motifs capable of being replaced for the remaining parts excluding the corresponding part. However, this brute-force-like approach requires considerable expertise and enormous costs due to the large size of the chemical space, such as drugs estimated to be in a range from 1030 to 1060.

To address this inefficiency, various computer-aided drug design methods, in particular, application programs based on artificial intelligence (AI) technology have been proposed. However, various conventional artificial intelligence-based molecular generation models are effective in generating molecules that satisfy certain chemical properties. In the meantime, they still have room for improvement to simultaneously achieve the generation of molecules with a similar structure to the source molecule.

### SUMMARY

According to an embodiment, a training method for a molecular generation model includes obtaining a training dataset including a source molecular model, a target molecular model whose structural similarity with the source molecular model exceeds a first threshold, and a negative molecular model whose structural similarity with one or more models of the source molecular model or the target molecular model is smaller than or equal to the first threshold, training the molecular generation model to adjust a distance between the source molecular model and the target molecular model based on the training dataset and a first loss function, and training the molecular generation model to adjust at least one distance of a distance between the source molecular model and the negative molecular model, and a distance between the target molecular model and the negative molecular model based on the training dataset and a second loss function different from the first loss function.

According to an embodiment, the training of the molecular generation model to adjust the distance between the source molecular model and the target molecular model based on the training dataset and the first loss function may include training the molecular generation model to decrease the distance between the source molecular model and the target molecular model based on the training dataset and the first loss function.

According to an embodiment, the training of the molecular generation model to adjust the at least one distance of the distance between the source molecular model and the negative molecular model, and the distance between the target molecular model and the negative molecular model based on the training dataset and the second loss function different from the first loss function may include training the molecular generation model to increase the at least one distance of the distance between the source molecular model and the negative molecular model, and the distance between the target molecular model and the negative molecular model, based on the training dataset and the second loss function different from the first loss function.

According to an embodiment, the training method may further include training the molecular generation model such that a molecular model whose structural similarity with the source molecular model exceeds a second threshold is output from the source molecular model, based on the training dataset and a reward function.

According to an embodiment, the training of the molecular generation model such that the molecular model whose structural similarity with the source molecular model exceeds the second threshold is output from the source molecular model, based on the training dataset and the reward function may include obtaining an output molecular model by entering the source molecular model into the molecular generation model, and calculating a positive weight or a negative weight associated with the output molecular model and assigning the positive weight or the negative weight to the molecular generation model, based on whether structural similarity between the output molecular model and the source molecular model exceeds the second threshold as a result of comparing the output molecular model and the source molecular model.

According to an embodiment, the calculating of the positive weight or the negative weight associated with the output molecular model and the assigning of the positive weight or the negative weight to the molecular generation model, based on whether the structural similarity between the output molecular model and the source molecular model exceeds the second threshold as the result of comparing the output molecular model and the source molecular model may include calculating the positive weight or the negative weight associated with the output molecular model and the assigning the positive weight or the negative weight to the molecular generation model, based on whether the structural similarity between the output molecular model and the source molecular model exceeds the second threshold, and whether a chemical property score of the output molecular model exceeds a chemical property score of the source molecular model as the results of comparing the output molecular model and the source molecular model.

According to an embodiment, the training of the molecular generation model such that the molecular model whose structural similarity with the source molecular model exceeds the second threshold is output from the source molecular model, based on the training dataset and the reward function may include training the molecular generation model such that a molecular model whose structural similarity with the source molecular model exceeds the second threshold and which has a chemical property score greater than a chemical property score of the source molecular model, is output from the source molecular model, based on the training dataset and the reward function.

According to an embodiment, a chemical property score of the target molecular model may be greater than a chemical property score of the source molecular model.

According to an embodiment, a computer program recorded in a computer-readable recording medium to perform the training method for a molecular generation model.

According to an embodiment, a training device for a molecular generation model includes a memory that stores data associated with the molecular generation model, and at least one processor connected to the memory and training the molecular generation model. The at least one processor includes instructions. the instructions, when executed, cause the at least one processor to obtain a training dataset including a source molecular model, a target molecular model whose structural similarity with the source molecular model exceeds a first threshold, and a negative molecular model whose structural similarity with one or more models of the source molecular model or the target molecular model is smaller than or equal to the first threshold, to train the molecular generation model to adjust a distance between the source molecular model and the target molecular model based on the training dataset and a first loss function, and to train the molecular generation model to adjust at least one distance of a distance between the source molecular model and the negative molecular model, and a distance between the target molecular model and the negative molecular model based on the training dataset and a second loss function different from the first loss function.

### BRIEF DESCRIPTION OF THE FIGURES

FIG. 1 is an architecture showing an operation of a molecular generation model, according to an embodiment of the present disclosure.

FIGS. 2A to 2C are architectures showing a part of a process of training a molecular generation model by using training datasets, according to an embodiment of the present disclosure.

FIG. 3 is an architecture showing another part of a process of training a molecular generation model by using an arbitrary input molecular model, according to an embodiment of the present disclosure.

FIG. 4 is a block diagram showing an internal configuration of a computing device 410, according to an embodiment of the present disclosure.

FIGS. 5A to 7D show results of evaluating success rates for several structural similarity thresholds ranging from 0.40 to 0.70, according to an embodiment of the present disclosure.

FIGS. 8A to 8D show results of an ablation experiment on a DRD2 benchmark dataset to demonstrate merits of the training method according to an embodiment of the present disclosure.

FIG. 9 shows a result of evaluating three indicators: property, improvement, and similarity for each trained model with or without the first loss function (a contractive loss) and the second loss function (a margin loss), according to an embodiment of the present disclosure.

FIG. 10 shows the result of evaluating the average structural similarity of loss functions (contractive & margin), according to an embodiment of the present disclosure.

FIGS. 11A and 11B show results of linear projection analysis of a molecular generation model, according to an embodiment of the present disclosure.

FIG. 12 shows the result of comparing success rates after 10,000 molecular models are trained and generated by using sorafenib as a source molecular model, according to an embodiment of the present disclosure.

FIG. 13 shows the visually identified result of generating a molecular model with a structure similar to sorafenib from a molecular generation model, according to an embodiment of the present disclosure.

FIGS. 14A and 14B show results of comparing binding energies by using AutoDock Vina to determine whether a hit candidate in the experimental example has a higher binding energy to ABCG2 than sorafenib, according to an embodiment of the present disclosure.

FIG. 15 shows a graphical representation of a 3D structure of a receptor-ligand complex using Chimera and a graphical representation of a 2D structure of a receptor-ligand complex using LigPlot Plus, to determine whether a hit candidate has as strong binding affinity for BRAF as sorafenib in an experimental example, according to one embodiment of the present disclosure.

FIG. 16 is a flowchart of a training method for a molecular generation model, according to an embodiment of the present disclosure.

### DETAILED DESCRIPTION

Hereinafter, details for implementing the present disclosure will be described in detail with reference to the accompanying drawings. However, in the following description, when there is a risk of unnecessarily obscuring the gist of the present disclosure, detailed descriptions of well-known functions or configurations will be omitted.

In the accompanying drawings, identical or corresponding components are assigned the same reference numerals. Moreover, in the description of embodiments below, descriptions of the same or corresponding components may be omitted to avoid redundancy. However, even though descriptions regarding components are omitted, it is not intended that such components are not included in any embodiment.

The above and other aspects, features and advantages of the present disclosure will become apparent from embodiments to be described in conjunction with the accompanying drawings. However, the present disclosure may be embodied in various different forms, and should not be construed as being limited only to the illustrated embodiments. Rather, these embodiments are provided as examples such that the present disclosure will be thorough and complete, and will fully convey the scope of the present disclosure to those skilled in the art.

Terms used in this specification will be briefly described, and the disclosed embodiments will be described in detail. Although certain general terms widely used in this specification are selected to describe embodiments in consideration of the functions thereof, these general terms may vary according to intentions of one of ordinary skill in the art, case precedents, the advent of new technologies, and the like. Terms arbitrarily selected by the applicant of the embodiments may also be used in a specific case. In this case, their meanings are given in the detailed description of the present disclosure. Hence, these terms used in the present disclosure may be defined based on their meanings and the contents of the present disclosure, not by simply stating the terms.

Expressions in the singular used in this specification include a plurality of expressions unless interpreted otherwise in context. A plurality of expressions includes expressions in the singular unless the context clearly dictates that the expression is plural. It will be understood that the terms “comprises,” “comprising,” “includes” and/or “including,” when used herein, specify the presence of stated elements and/or components, but do not preclude the presence or addition of one or more other elements and/or components.

The term “unit” used herein may refer to software or hardware, and the “unit” may perform some functions. However, the “unit” may be not limited to software or hardware. The “unit” may be configured to exist in an addressable storage medium or may be configured to play one or more processors. Therefore, as an example, “units” may include at least one of various elements such as software elements, object-oriented software elements, class elements, and task elements, processes, functions, attributes, procedures, subroutines, program code segments, drivers, firmware, microcodes, circuits, data, databases, data structures, tables, arrays, or variables. Functions provided in “units” and components may be combined into a smaller number of “units” and components or may be divided into additional “units” and components.

According to an embodiment of the present disclosure, the ‘unit’ may be implemented with a processor and a memory. The ‘processor’ needs to be interpreted broadly to include a general-purpose processor, a central processing unit (CPU), a microprocessor, a digital signal processor (DSP), a controller, a microcontroller, state machine, or the like. In some environments, the ‘processor’ may also refer to an application-specific integrated circuit (ASIC), a programmable logic device (PLD), a field programmable gate array (FPGA), or the like. The ‘processor’ may also refer to a combination of processing devices, for example, a combination of a DSP and a microprocessor, a combination of a plurality of microprocessors, a combination of one or more microprocessors combined with a DSP core, or any other combination of configurations. Moreover, the ‘memory’ needs to be broadly interpreted to include any electronic component capable of storing electronic information. The ‘memory’ may refer to various types of processor-readable media such as a random access memory (RAM), a read-only memory (ROM), a non-volatile random access memory (NVRAM), a programmable read-only memory (PROM), an erasable-programmable read-only memory (EPROM), an electrically erasable PROM (EEPROM), a flash memory, a magnetic or optical data storage device, a register, and the like. As long as a processor is capable of reading out information from and/or writing information to memory, it is referred to as a “state where the memory communicates with the processor in a wired or wireless manner”. The memory integrated into the processor is in electronic communication with the processor.

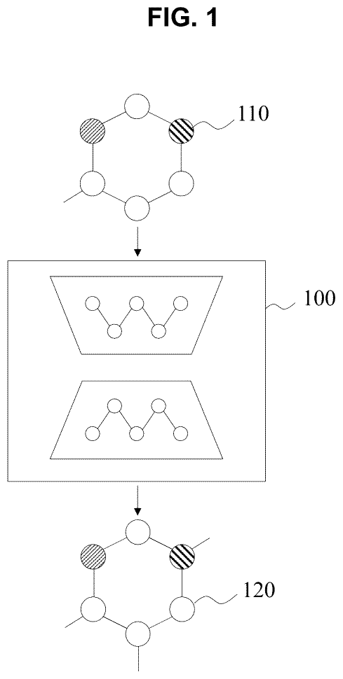

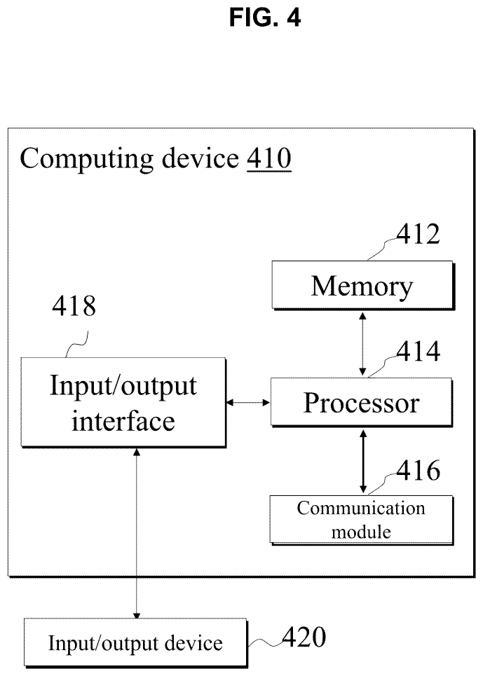

FIG. 1 is an architecture showing an operation of a molecular generation model 100, according to an embodiment of the present disclosure. As shown in drawings, the molecular generation model 100 may obtain a final molecular model 120 from a source molecular model 110 that is entered. Here, the final molecular model 120 may refer to a molecular model whose physical and/or chemical properties have been modified in the source molecular model 110. In the meantime, operations related to the molecular generation model 100 are performed by at least one processor of a computing device, and a hardware configuration of the computing device is described in detail later with reference to FIG. 4.

The source molecular model 110 may be structurally similar to the final molecular model 120. In other words, the molecular generation model 100 may be configured such that the structural similarity between the source molecular model 110 and the final molecular model 120 exceeds a predetermined threshold. In this case, the structural similarity may be measured depending on the tanimoto similarity measurement method.

The source molecular model 110 may not have a similar chemical property score to the final molecular model 120. In detail, the chemical property score of the final molecular model 120 may be configured to be greater than the chemical property score of the source molecular model 110. This may mean that the chemical properties of the final molecular model 120 are improved compared to the source molecular model 110. Moreover, the chemical property score may mean the degree of a specific chemical reaction of a molecular model such as the source molecular model 110 and/or the final molecular model 120. For example, when the molecular model is a drug, the chemical property score may mean the extent to which the molecular model reacts with specific cells in the human body.

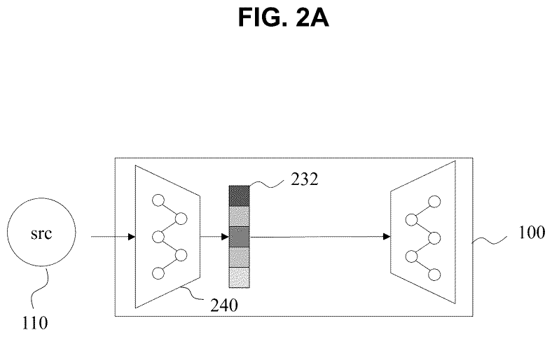

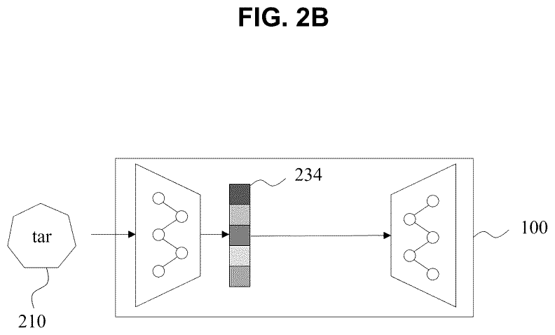

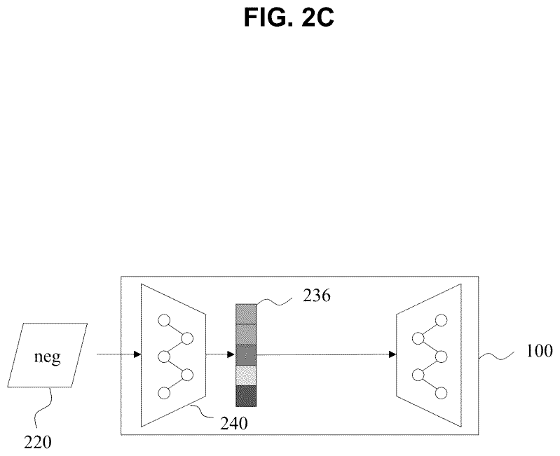

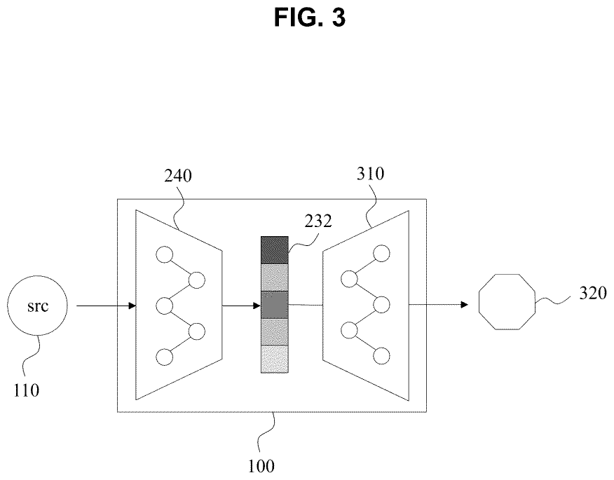

The processor may train the molecular generation model 100 such that the chemical properties are improved from the source molecular model 110 and the final molecular model 120 with a similar molecular structure is output. To this end, the processor may train the molecular generation model 100 such that the molecular generation model 100 trains the chemical properties and molecular structure of the input molecular model (here, the source molecular model 110) by using a training dataset including a plurality of molecular structure model samples. As such, in FIGS. 2A to 2D and 3, a process in which the processor generates a training dataset and trains the molecular generation model 110 by using the training dataset is described in detail later.

FIGS. 2A to 2C are architectures showing a part of a process of training the molecular generation model 100 by using training datasets 110, 210, and 220, according to an embodiment of the present disclosure. As shown in drawings, a training dataset may include at least one of the source molecular model 110, a target molecular model 210, and a negative molecular model 220. In this case, the target molecular model 210 may be configured such that structural similarity with the source molecular model 110 exceeds a first threshold. Furthermore, the negative molecular model 220 may be configured such that the structural similarity with the source molecular model 110 is smaller than or equal to the first threshold and the structural similarity with the target molecular model 210 is smaller than or equal to the first threshold. Additionally or alternatively, the negative molecular model 220 may be configured such that the structural similarity with the source molecular model 110 is smaller than or equal to a second threshold smaller than the first threshold, and the structural similarity with the target molecular model 210 is smaller than or equal to the second threshold less than the first threshold.

A processor may obtain the training datasets 110, 210, and 220. Then, the processor may train the molecular generation model 100 to adjust a distance between the source molecular model 110 and the target molecular model 210 based on the training datasets 110, 210, and 220 and a first loss function. In detail, the processor may train the molecular generation model 100 to decrease the distance between the source molecular model 110 and the target molecular model 210 based on the training datasets 110, 210, and 220 and the first loss function. For example, the processor may obtain a first vector 232 by entering the source molecular model 110 into the molecular generation model 100. Besides, the processor may obtain a second vector 234 by entering the target molecular model 210 into the molecular generation model 100. Then, the processor may decrease the distance between the first vector 232 and a second vector 234. In other words, the processor may train the structural similarity between the source molecular model 110 and the target molecular model 210 based on the first loss function.

The processor may train the molecular generation model 100 to adjust the distance between the source molecular model 110 and the negative molecular model 220 based on the training datasets 110, 210, and 220 and a second loss function different from the first loss function. In detail, the processor may train the molecular generation model 100 to decrease the distance between the source molecular model 110 and the negative molecular model 220 based on the training datasets 110, 210, and 220 and the second loss function. For example, the processor may obtain a third vector 236 by entering the negative molecular model 220 into the molecular generation model 100. Then, the processor may increase the distance between the first vector 232 and the third vector 236. Accordingly, the distance between the first vector 232 and the third vector 236 may be configured to be farther than the distance between the first vector 232 and the second vector 234. In other words, the processor may train the structural dissimilarity between the source molecular model 110 and the negative molecular model 220 based on the second loss function.

The processor may train the molecular generation model 100 to adjust the distance between the target molecular model 210 and the negative molecular model 220 based on the training datasets 110, 210, and 220 and the second loss function. In detail, the processor may train the molecular generation model 100 to adjust the distance between the target molecular model 210 and the negative molecular model 220 based on the training datasets 110, 210, and 220 and the second loss function. For example, the processor may increase the distance between the second vector 234 and the third vector 236. Accordingly, the distance between the second vector 234 and the third vector 236 may be configured to be farther than the distance between the first vector 232 and the second vector 234. In other words, the processor may train the structural dissimilarity between the target molecular model 210 and the negative molecular model 220 based on the second loss function.

In the meantime, as shown in drawings, the first vector 232, the second vector 234, and the third vector 236 may be obtained by entering the source molecular model 110, the target molecular model 210, and the negative molecular model 220 into a first module 240 of the molecular generation model 100, respectively In this case, the first module 240 may include at least one encoder.

FIG. 3 is an architecture showing another part of a process of training the molecular generation model 100 by using an arbitrary input molecular model (here, the source molecular model 110), according to an embodiment of the present disclosure. A process of training the molecular generation model 100 in FIG. 3 may be performed after a process of training the molecular generation model 100 in FIGS. 2A to 2C. Additionally or alternatively, the process of training the molecular generation model 100 may be performed at least partially in parallel with the process of training the molecular generation model 100 of FIGS. 2A to 2C.

A processor may train the molecular generation model 100 such that a molecular model whose structural similarity with any molecular model exceeds a predetermined threshold is output from any input molecular model, based on a training dataset (e.g., the training datasets 110, 210, and 220) and a reward function. In other words, the reward function may strengthen training of the structural similarity and chemical properties of the output molecular model by assigning a positive weight to the molecular generation model 100 based on the physical structure and/or chemical properties of the molecular model (here, an output molecular model 320) output from the molecular generation model 100. Furthermore, the reward function may strengthen training of the structural dissimilarity and chemical properties of the output molecular model by assigning a negative weight to the molecular generation model 100 based on the physical structure and/or chemical properties of the molecular model output from the molecular generation model 100. Hereinafter, a process of training the molecular generation model 100 will be described in detail by using the case where the source molecular model 110 is entered.

First of all, the processor may obtain a vector corresponding to the molecular model by entering any molecular model included in the training dataset into the molecular generation model 100. For example, the processor may obtain the first vector 232 by entering the source molecular model 110 included in the training dataset into the molecular generation model 100 (in particular, the first module 240). Then, the processor may obtain the output molecular model 320 by entering the obtained first vector 232 into a second module 310.

The processor may compare the obtained output molecular model 320 with the source molecular model 110. In particular, the processor may calculate structural similarity between the output molecular model 320 and the source molecular model 110. Additionally or alternatively, the processor may calculate the chemical property score of the output molecular model 320.

When the structural similarity between the output molecular model 320 and the source molecular model 110 exceeds a predetermined threshold, the processor may calculate a positive weight associated with the output molecular model 320. In this case, the positive weight associated with the output molecular model 320 may be determined based on the structural similarity and/or chemical property score of the output molecular model 320. Then, the processor may assign the calculated positive weight to the molecular generation model 100. On the other hand, when the structural similarity between the output molecular model 320 and the source molecular model 110 is smaller than or equal to the predetermined threshold, the processor may calculate a negative weight associated with molecular model 110.

In the meantime, in calculating the positive weight and/or the negative weight, the processor may simultaneously consider the structural similarity and the chemical property score. For example, when the chemical property score of the output molecular model 320 exceeds a predetermined threshold, and the structural similarity between the output molecular model 320 and the source molecular model 110 exceeds a predetermined threshold, the processor may calculate the positive weight based on the structural similarity and/or chemical property score of the output molecular model 320 and may assign the positive weight to the molecular generation model 100. For example, when the chemical property score of the output molecular model 320 is smaller than the predetermined threshold, and the structural similarity between the output molecular model 320 and the source molecular model 110 is smaller than or equal to the predetermined threshold, the processor may calculate the negative weight based on the structural similarity and/or chemical property score of the output molecular model 320 and may assign the negative weight to the molecular generation model 100. Likewise, even when each of the chemical property score and structural similarity is smaller than the corresponding threshold, the processor may calculate the negative weight and may assign the negative weight to the molecular generation model 100.

FIG. 4 is a block diagram showing an internal configuration of a computing device 410, according to an embodiment of the present disclosure. Here, the computing device 410 may refer to a device for executing a training method for a molecular generation model according to an embodiment of the present disclosure. The computing device 410 may be any computing device capable of executing programs for machine learning and capable of wired/wireless communication, and may include a desktop, a smartphone, a tablet PC, a laptop, or the like.

The computing device 410 may include a memory 412, a processor 414, a communication module 416, and an input/output interface 418. Although not shown in FIG. 4, the computing device 410 may be configured to exchange information and/or data over a network by using the communication module 416. Moreover, an input/output device 420 may be configured to input information and/or data to the computing device 410 through the input/output interface 418 or to output information and/or data generated from the computing device 410.

The memory 412 may include any computer-readable recording medium. According to an embodiment, the memory 412 may include a permanent mass storage device such as a random access memory (RAM), a read only memory (ROM), a disk drive, a solid state drive (SSD), a flash memory, or the like. For another example, the permanent mass storage device such as ROM, SSD, flash memory, or disk drive may be included in the computing device 410 as a permanent storage device separate from the memory. Also, the memory 412 may store an operating system and at least one program code (e.g., a code for executing a program for machine learning installed and running in the computing device 410).

These software components may be loaded from a computer-readable recording medium independent of the memory 412. Such the separate computer-readable recording medium may include a recording medium capable of being directly connected to the computing device 410 and an external server, and may include, for example, a computer-readable recording medium such as a floppy drive, a disk, a tape, a DVD/CD-ROM drive, and a memory card. For another example, the software components may be loaded into the memory 412 through the communication module 416, not the computer-readable recording medium. For example, at least one program may be loaded into the memory 412 based on a computer program installed by files provided by developers or a file distribution system, which distributes a file for installing an application, over the network.

The processor 414 may be configured to process an instruction of a computer program by performing basic arithmetic, logic, and input and output operations. The instruction may be provided to the processor 414 by the memory 412 or the communication module 416. For example, the processor 414 may be configured to execute instructions received depending on a program code stored in a recording device such as the memory 412.

The communication module 416 may provide a configuration or function that allows the computing device 410 and the external server to communicate with each other over the network. The computing device 410 and/or the external server may provide a configuration or function for communicating with another user terminal or another system (e.g., a separate cloud system, etc.). For example, under the control of the communication module 416, a request or data generated by the processor 414 of the computing device 410 depending on a program code stored in a recording device such as the memory 412 may be delivered to the external server over the network. Inversely, a control signal or command provided under the control of a processor of the external server may be received by the computing device 410 through the communication module 416 of the computing device 410 over the network.

The input/output interface 418 of the computing device 410 may be a means for interaction with the input/output device 420. For another example, the input/output interface 418 may be a means for an interface with a device, in which configurations or functions for performing an input and an output are integrated into one, such as a touch screen. In this case, the input/output device 420 may include an input device such as a camera including an audio sensor and/or an image sensor, a keyboard, a microphone, and a mouse. Moreover, the input/output device 420 may include an output device such as a display, a speaker, a haptic feedback device, and the like.

In FIG. 4, the input/output device 420 is shown not to be included in the computing device 410, but is not limited thereto. For example, the input/output device 420 and the computing device 410 may be composed of one device. Furthermore, in FIG. 4, the input/output interface 418 is shown as an element configured separately from the processor 414, but is not limited thereto. For example, the input/output interface 418 may be configured to be included in the processor 414.

The computing device 410 may include more components than those of FIG. 4. However, there is no need to clearly illustrate most conventional components. According to an embodiment, the computing device 410 may be implemented to include at least part of the input/output device 420 described above. In addition, the computing device 410 may further include other components such as a transceiver, a global positioning system (GPS) module, a camera, various sensors, and a database. For example, when the computing device 410 is a smartphone, the computing device 410 may include components generally included in the smartphone. For example, various components, such as an acceleration sensor, a gyro sensor, a microphone module, a camera module, various physical buttons, a button using a touch panel, input/output ports, a vibrator for vibration, and the like may be implemented to be further included in the computing device 410.

While a program for machine learning is running on each of the computing device 410 and the external server, the processor 414 may receive numerical data, texts, images, videos, and the like, which are entered or selected through the input/output device 420 connected to the input/output interface 418, and may store the received numerical data, texts, images, and/or video in the memory 412 or provide the stored result to each other through the communication module 416 and a network.

The processor of the external server may be configured to manage, process, and/or store information and/or data received from a plurality of user terminals and/or a plurality of external systems. According to an embodiment, the processor may manage, process, and/or store a user input, which is received from the computing device 410, and data according to the user input. Additionally or alternatively, the processor may be configured to store and/or update a program for executing an algorithm used in machine learning of the computing device 410 from a separate cloud system, database, or the like, which is connected to the network.

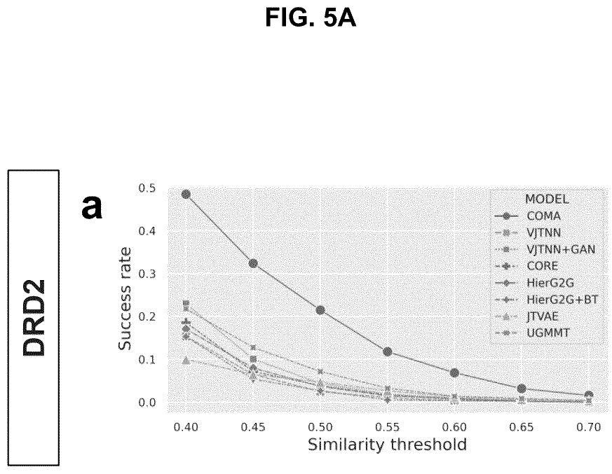

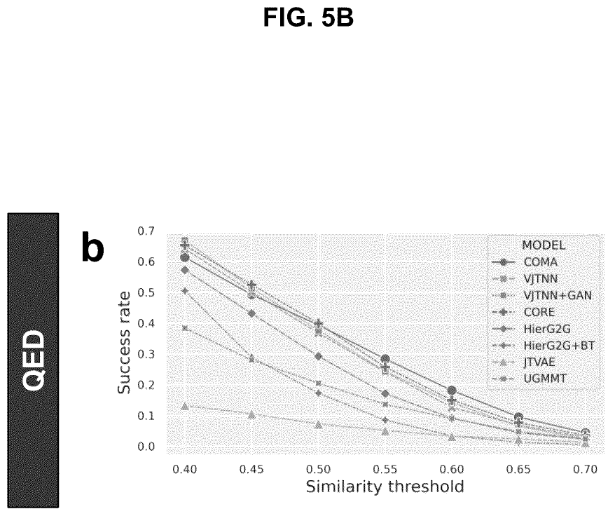

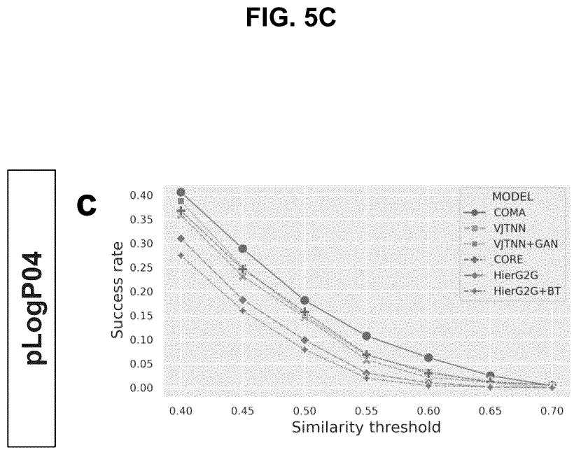

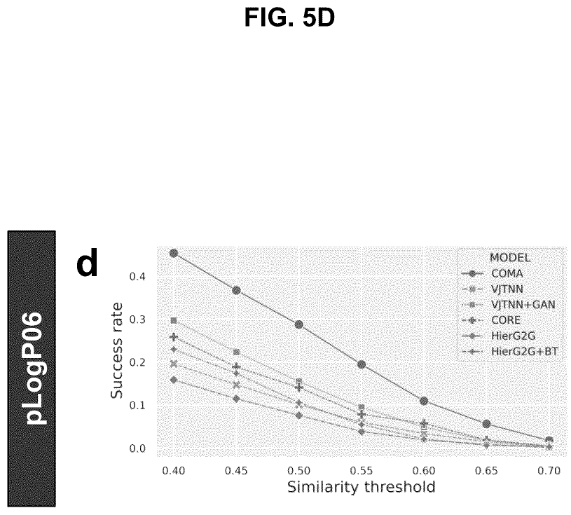

Hereinafter, experimental examples performed to implement a training method for the molecular generation model (indicated by “COMA” in FIGS. 5A to 15) according to an embodiment of the present disclosure are described in detail.

### Overview of Molecular Generation Model

The molecular generation model according to an embodiment of the present disclosure may be a variational autoencoder (VAE) based on a gated recurrent unit (GRU) for encoding and decoding a SMILES string, and may represent the molecular structure using ASCII codes. Here, each of the ASCII codes may represent atoms included in a molecular structure, the type of bond between the atoms, and/or a bond structure (e.g., a branch structure, a ring structure, or the like).

The encoder (i.e. first module) of the molecular generation model may insert two molecules with similar structures at points close to each other in the latent space based on the first loss function, but may place two molecules with different structures as far as possible in the latent space based on the second loss function. In other words, the decoder of the generation molecular model is trained to generate a valid SMILES string from the potential vector (e.g., the first vector 232 in FIGS. 2A to 2C) output from the encoder. In addition, reinforcement learning using a reward function may be applied to selectively generate a SMILES string with improved chemical properties than the input molecular model (e.g., the source molecular model 110 in FIG. 3).

### Performance Evaluation

Four benchmark datasets DRD2, QED, p Log P04, and p Log P06 are used to evaluate the performance of the molecular generation model. Training using DRD2 aims to generate a new molecule that is more active in a dopamine receptor D2 than the source molecular model under the condition that tanimoto similarity is 0.4 or more. Training using QED aims to generate a new molecule that is more similar to a (existing) drug than the source molecular model under the condition that tanimoto similarity is 0.4 or more. For QED score, a range is [0,1]. As the value is greater, the similarity with the drug is higher. Lastly, p Log P04 and p Log P06 tasks aim to improve the score of penalty log P by setting each of 0.4 and 0.6 as a structural similarity threshold with the source molecular model. Here, the score of penalty log P represents a value obtained by subtracting a size value of a ring in a molecular structure and a synthetic accessibility score from the score of log P.

### Comparison Model

The molecular structure comparison model according to an embodiment of the present disclosure is compared with seven latest models: JTVAE, VJTNN, VJTNN+GAN, CORE, HierG2G, HierG2G+BT, and UGMMT. JTVAE represents a graph-based molecular generation model that optimizes molecular properties by using a Bayesian optimization method. VJTNN is the latest version of JTVAE model having an added neural attention function, and VJTNN+GAN is the latest version of model with adversarial training. CORE is an improved version of VJTNN+GAN that generates molecules by using a copy-and-refine strategy. HierG2G is a graph-based generation model using a hierarchical encoding method. HierG2G+BT is an improved version of HierG2G that adds a back-translation step for data augmentation. UGMMT is a SMILES-based generation model trained by using an unsupervised learning method.

### Evaluation Indicators

Molecular generation models and other models are evaluated by using various evaluation indicators for structure-constrained molecular generation. First of all, all models are trained with the training dataset of each benchmark task, molecules are generated 20 times for each source molecule in the test dataset, and the generated molecules are evaluated by using 7 indicators.
Validity: a ratio of valid SMILES strings generated from test data.
Novelty: a ratio of valid SMILES strings that are not in the training data.
Property: an average property score of a valid SMILES string.
Improvement: an average of property score differences between generated SMILES string and source SMILES string.
Similarity: an average of tanimoto structural similarity between generated SMILES string and source SMILES string.
Diversity: an average of tanimoto pairwise dissimilarity between generated SMILES strings.
Success rate: a ratio of valid new SMILES strings that satisfy both improvement in chemical properties (here, drug properties) and structural similarity criteria.

### Comparison of Success Rate

FIGS. 5A to 7D show results of evaluating success rates for several structural similarity thresholds ranging from 0.40 to 0.70, according to an embodiment of the present disclosure. A success rate indicates the most important measure in measuring the amount of valid molecules, which are generated by a model and which simultaneously satisfy the three constraints: novelty, improvement in chemical properties, and structural similarity.

Referring to FIGS. 5A to 7D, it is identified that a molecular generation model according to an embodiment of the present disclosure may have the performance better than or equal to the performance of a basic model under various threshold conditions, and the molecular generation model may generate molecules that satisfy other conditions under structural similarity constraints 0.55 to 0.70 better than the basic model.

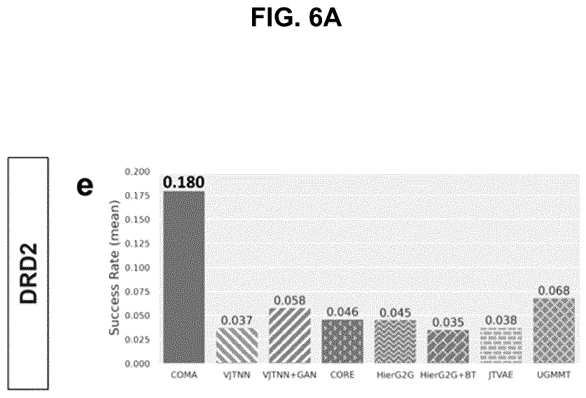

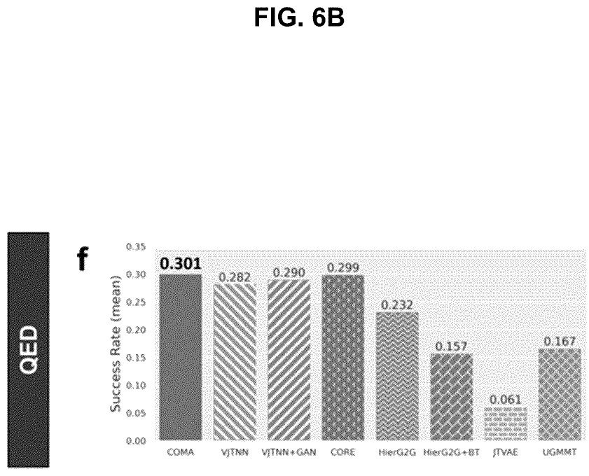

Referring to FIGS. 6A to 6D, it is identified that the average success rate for a threshold of structural similarity is calculated for quantitative comparison, and average scores of the molecular generation model according to an embodiment of the present disclosure for DRD2, QED, p Log P04, and p Log P06 respectively correspond to 0.180, 0.301, 0.154, and 0.213. It is identified that it is a more suitable model for generating molecules with structure constraints because the score of the molecular generation model is higher by a range from 0.002 to 0.240, compared to the latest model, when the basic model is compared with the molecular generation model.

### Overall Performance

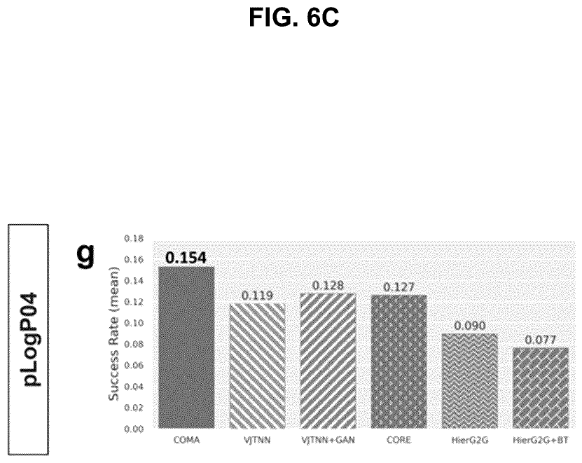

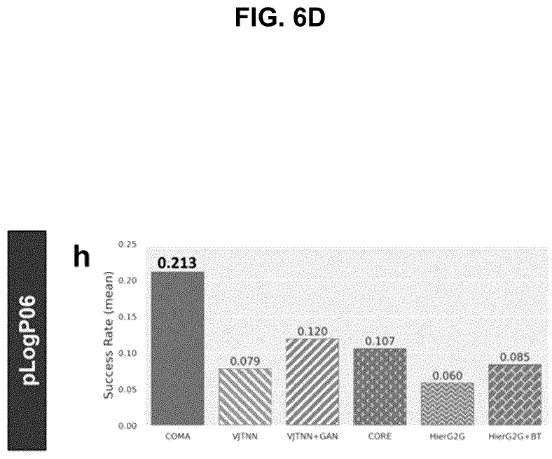

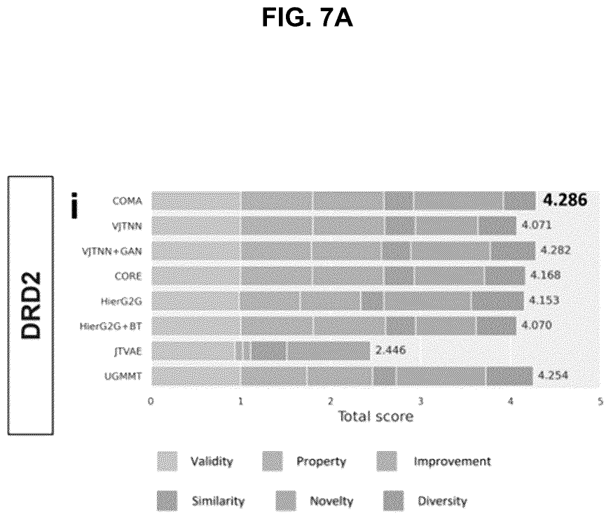

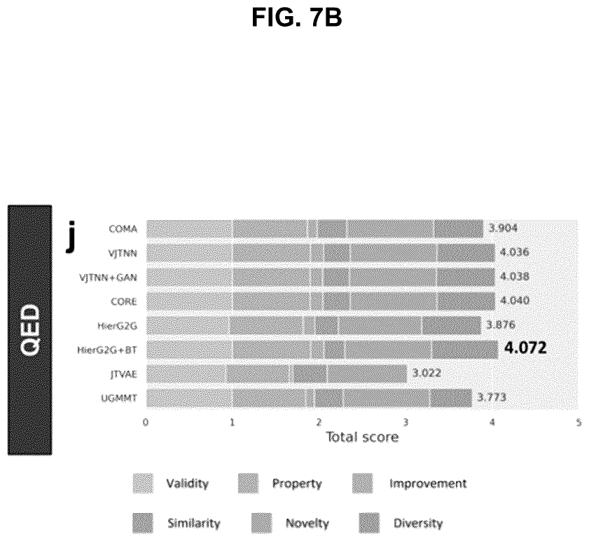

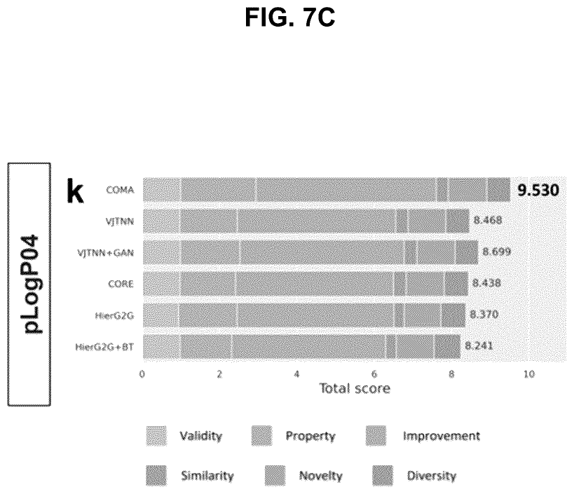

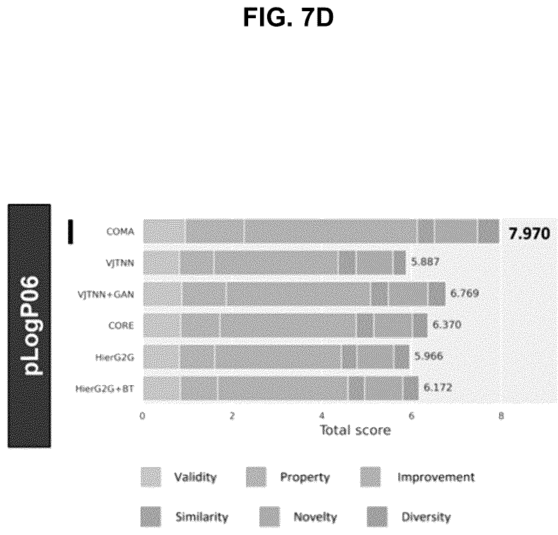

FIGS. 7A to 7D show that the remaining six indicators indicate characteristics of a molecular generation model according to an embodiment of the present disclosure and other models. The validity and novelty of the molecular generation model outperformed the baseline model on all benchmark datasets. In DRD2 and QED tasks, JTVAE has better structural similarity than the molecular generation model, but does not improve the chemical property score at the same time, resulting in a low success rate. The total validity, property, improvement, similarity, novelty, and diversity score of each model are calculated for overall evaluation. The molecular generation model excluding QED shows the highest score. Accordingly, it is identified that the above-mentioned success rate analysis is matched. The experimental results demonstrate that the training method according to an embodiment of the present disclosure using a first loss function, a second loss function, and a reward function is effective in generating molecules with structural constraints.

### Ablation Study on First Loss Function and Second Loss Function

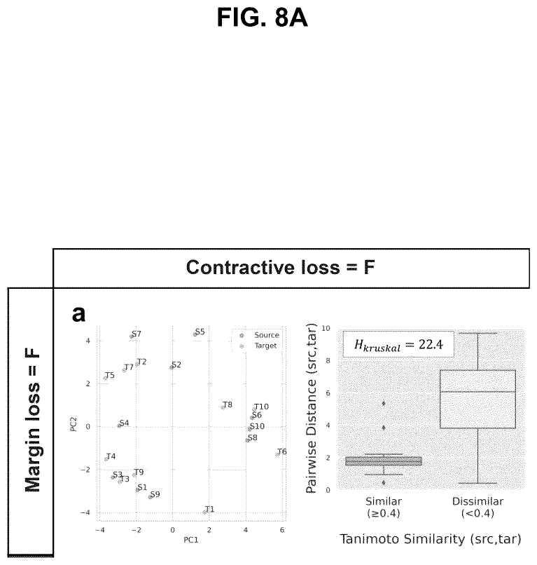

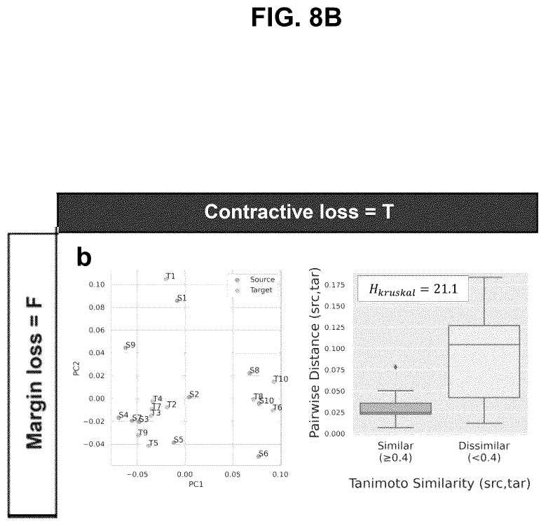

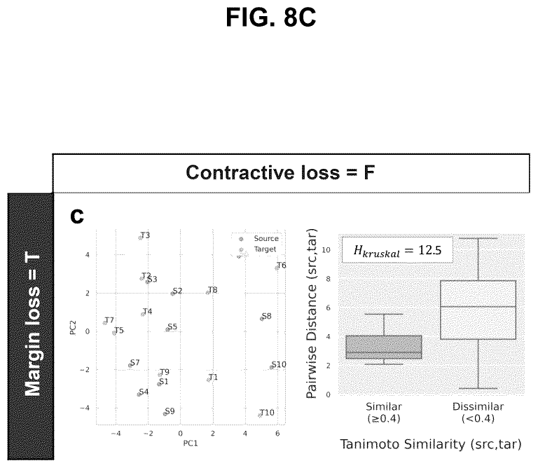

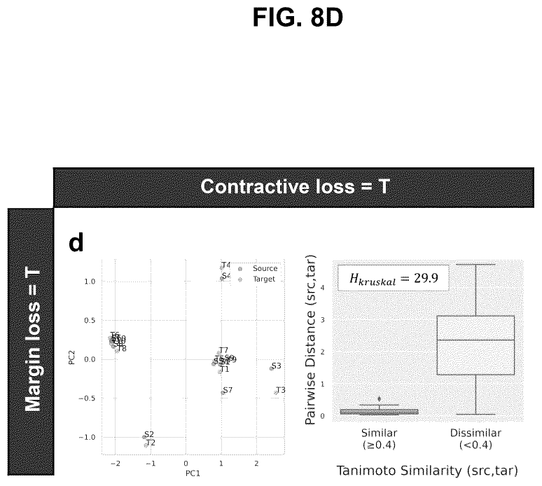

FIGS. 8A to 8D show results of an ablation experiment on a DRD2 benchmark dataset to demonstrate merits of the training method according to an embodiment of the present disclosure. As shown in drawings, high structural similarity may be achieved through training using both a first loss function (here, referred to as a “contractive loss”) and a second loss function (here, referred to as a “margin loss”). It is identified that training using both the first loss function and the second loss function results in statistically significant improvement in structural similarity by using Kruskal-Wallis H test.

### Performance Comparison

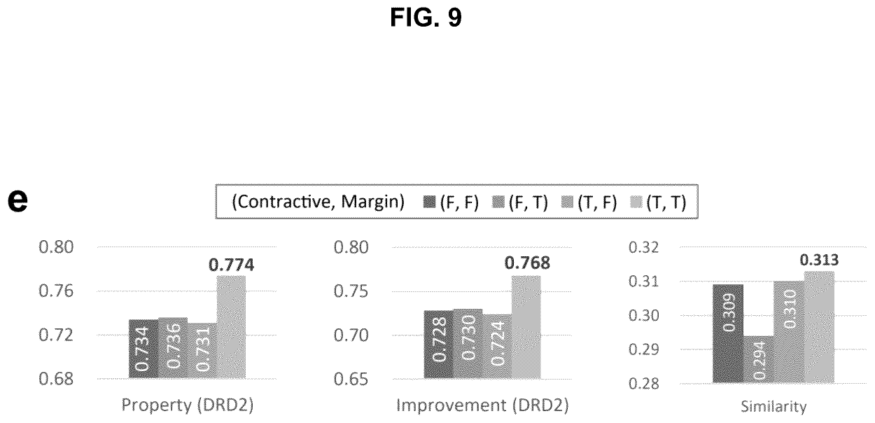

FIG. 9 shows a result of evaluating three indicators: property, improvement, and similarity for each trained model with or without the first loss function (here, referred to as a “contractive loss”) and the second loss function (here, referred to as a “margin loss”), according to an embodiment of the present disclosure. There is no noticeable difference in similarity, but high properties and improved scores are observed only when both the first and second loss functions are used. These results indicate that the first loss function and the second loss function play an important role in the generation of structurally constrained molecules.

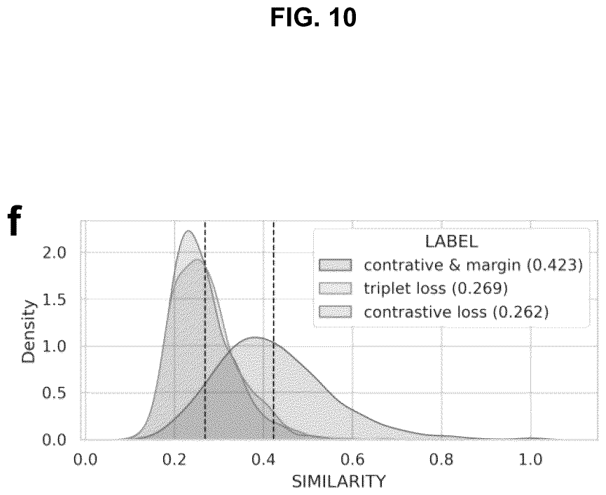

FIG. 10 shows a result of evaluating the average structural similarity of loss functions (here, referred to as “contractive & margin”), according to an embodiment of the present disclosure. It is identified that the combination of loss functions outperforms the conventional triplet loss and contrastive loss in a similar molecule generation task of DRD2 dataset. It is identified that the molecular generation model generates target molecules with average similarity of 0.423 with respect to the source molecular model. On the other hand, it is identified that the molecular generation model generates target molecules with average similarity of 0.269 and 0.262, when the conventional triplet loss and contrastive loss are used, respectively.

### Latent Space Analysis

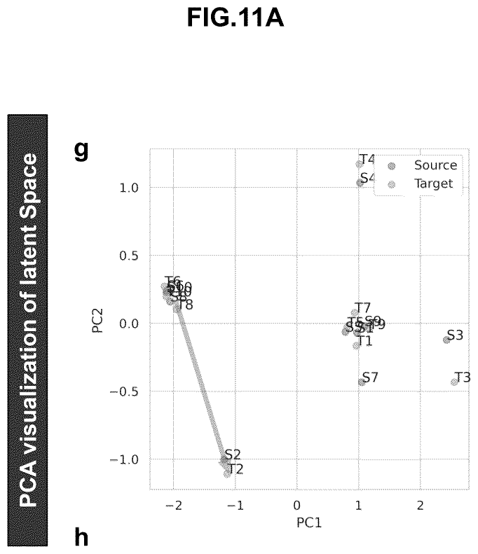

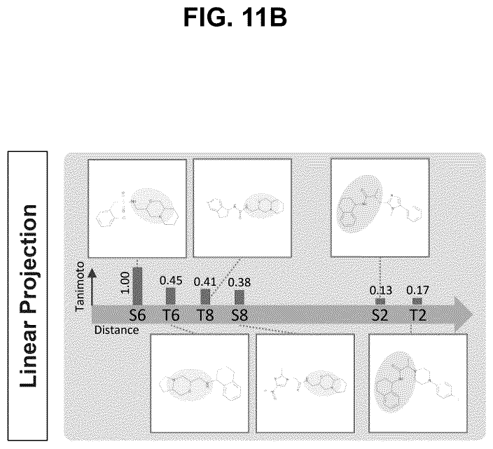

FIGS. 11A and 11B show results of linear projection analysis of a molecular generation model, according to an embodiment of the present disclosure. A molecular generation model improves performance in terms of structural similarity by using a first loss function and a second loss function. To this end, in the latent space of the molecular generation model, molecules with similar structures are designed to be close to each other, and molecules structurally different from each other are designed to be spaced apart from each other. The linear projection analysis uses the same data as data used in the statistical analysis described above. A point S6 is selected in latent space, and an arrow is drawn in a random direction starting from the point. The molecular structure corresponding to six points on the arrow is compared. The comparison result indicates that a point adjacent to the starting point has high tanimoto similarity, and a point far away from the starting point has a low similarity score. Accordingly, it is identified that the proposed method is as effective as intended.

### Specific Experimental Examples: Drug Discovery for Sorafenib Resistance

Structure-constrained molecule generation may be used to generate new molecules similar to molecules in existing drugs and to discover drug candidates for patients who are resistant to chemotherapy with drugs. The drug candidates may be obtained by reducing chemical properties associated with drug resistance without losing the pharmacophore properties of the existing drugs. In the present experiment, a molecular generation model is applied to sorafenib, which is a target anticancer drug for hepatocellular carcinoma (HCC), to improve the therapeutic effect of chemotherapy in patients with sorafenib-resistant liver cancer.

### Association Between Sorafenib Resistance and ABC Transporter

Sorafenib is an inhibitor of protein kinases that inhibits cell proliferation and angiogenesis in tumor cells in a Raf/Mek/Erk path. Due to the moderate therapeutic effectiveness of sorafenib and hidden drug resistance, the discovery of new drug candidates capable of being used as alternatives to sorafenib corresponds to an important research challenge. One of the suspected mechanisms associated with sorafenib resistance is the ATP-binding cassette (ABC) transporter, which pulls the drug out of the cell. Because multi-targeted tyrosine kinase inhibitors (TKIs), including sorafenib, act as substrates for ABC transporters, the ABC transporter is analyzed to remove sorafenib from HCC tumor cells before it may bind to therapeutic target proteins. Accordingly, when the binding affinity of sorafenib to the ABC transporter proteins is reduced without loss of affinity for therapeutic target proteins of the sorafenib, sorafenib resistance in hepatocellular carcinoma patients is mitigated while the effectiveness of chemotherapy is increased.

### Optimization of Binding Affinity for ABCG2

To perform proof-of-concept of the molecular generation model for discovery of hits similar to sorafenib, the goal is to preserve the substructure of sorafenib while the binding affinity score for the protein of ABC subfamily G member 2 (ABCG2) is reduced without losing the affinity for serine/threonine-protein kinase B-raf (BRAF) which is the target kinase of sorafenib, in this experiment. To this end, about 16,000 SMILES strings are selected from ChEMBL database, and the molecular generation model and a training dataset for UGMMT are created. Here, the UGMMT is selected because the UGMMT is the latest SMILES-based model.

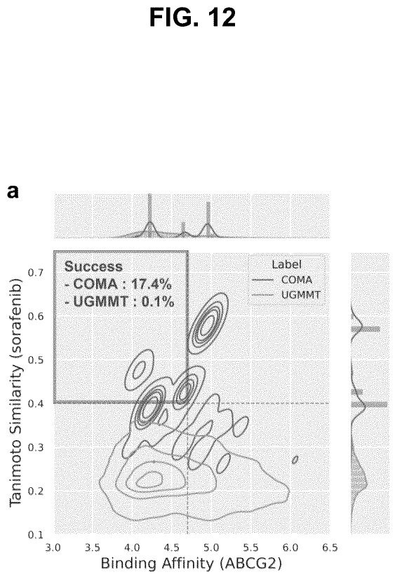

FIG. 12 shows the result of comparing success rates after 10,000 molecular models are trained and generated by using sorafenib as a source molecular model, according to an embodiment of the present disclosure. The success rate is defined as the ratio of new molecular models that satisfy a target molecular condition (tanimoto similarity >0.4 and affinity score for ABCG2<4.7). The molecular generation model (here, COMA) had a high success rate (0.174). On the other hand, UGMMT has a low success rate (0.001). Although the UGMMT reduces binding affinity for ABCG2 more than the molecular generation model, the UGMMT shows a low success rate because it failed to generate a molecule similar to sorafenib.

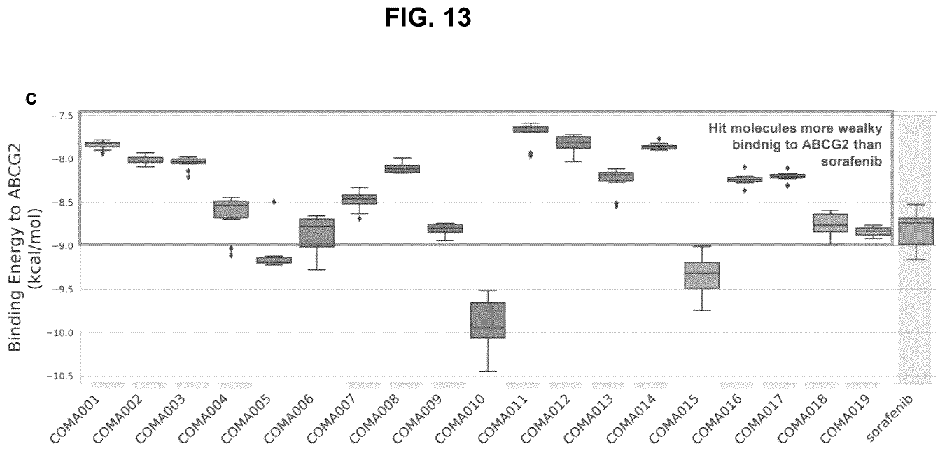

FIG. 13 shows the visually identified result of generating a molecular model with a structure similar to sorafenib from a molecular generation model, according to an embodiment of the present disclosure. It is identified that all molecules generated by the molecular generation model satisfy a target molecule condition (tanimoto similarity >0.4 and affinity score for ABCG2<4.7).

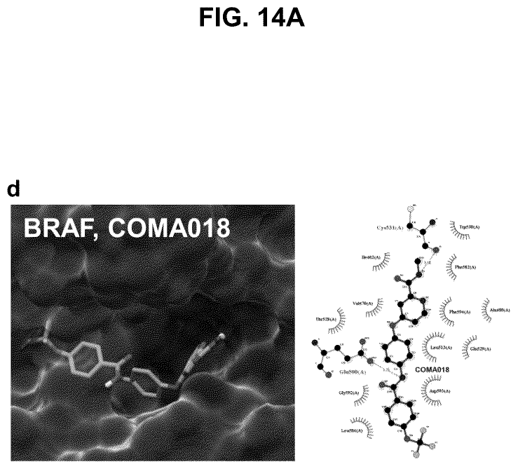

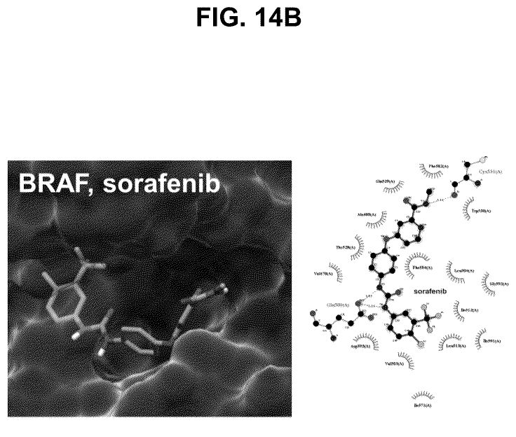

FIGS. 14A and 14B show results of comparing binding energies by using AutoDock Vina to determine whether a hit candidate in the experimental example has a higher binding energy to ABCG2 than sorafenib, according to an embodiment of the present disclosure. Docked poses are evaluated by using AutoDock Vina 1.2.3 and visualized by using Chimera 1.16 and LigPlot Plus 2.2.5. To prepare ligand, 19 unique molecules out of 10,000 molecules generated by the molecular generation model are first extracted through redundant molecular model removal, and three-dimensional (3D) coordinates of molecules are generated by using Open Babel 3.1.1. Then, the molecule is protonated at pH 7.4, and the pdbqt file is created by using meeko 0.3.0, which a Python library for AutoDock. To prepare ABCG2 and BRAF receptors, a 3D structure file including 6VXH and 1 UWH are downloaded from a PDB database for ABCG2 and BRAF, and hydrogens are identified by using ADFR software 1.0. Chimera is used to define the center and size of a box, and AutoDock Vina is executed to generate 20 poses per receptor-ligand pair. Then, the best pose with the highest binding energy score per receptor-ligand pair is selected and compared with sorafenib. Referring to FIGS. 14A and 14B, it may be seen that 15 molecules have higher binding energy for ABCG2 than sorafenib. Accordingly, these molecules may be hit candidate molecules as alternatives to sorafenib.

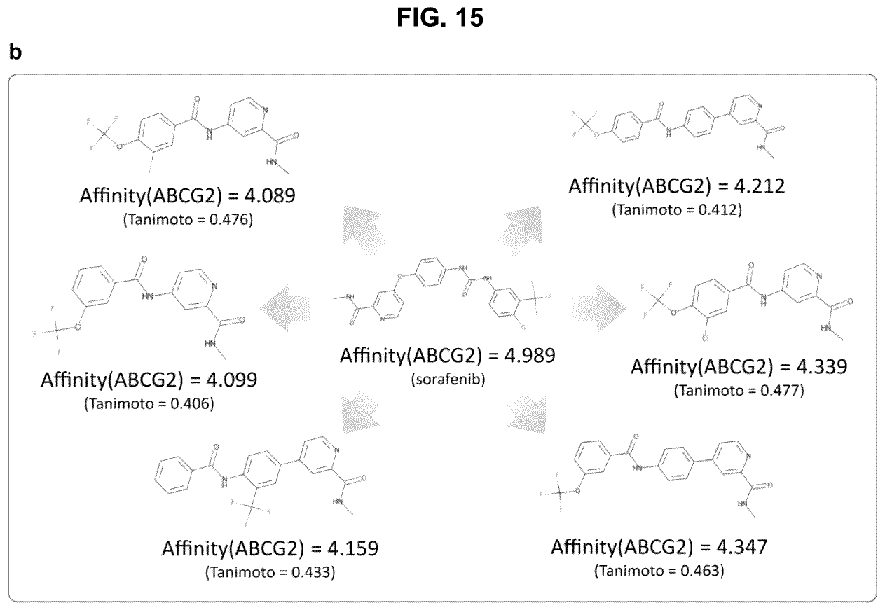

FIG. 15 shows a graphical representation of a 3D structure of a receptor-ligand complex using Chimera and a graphical representation of a 2D structure of a receptor-ligand complex using LigPlot Plus, to determine whether a hit candidate has as strong binding affinity for BRAF as sorafenib in an experimental example, according to one embodiment of the present disclosure. Referring to FIG. 15, it may be seen that hit candidate molecules fit well with a binding pocket for sorafenib in BRAF. Moreover, it is identified that the generated hit candidate molecule and sorafenib have common interatomic contacts based on van der Waals radii using structural analysis tools and default parameters. Furthermore, it is identified through a 2D plot drawn by LigPlot Plus that a molecule has hydrogen bonds with amino acid residues including Glu500 (A) and Cys531 (A) and interacts with sorafenib in BRAF.

### Synthetic Accessibility Evaluation

The synthetic feasibility of the molecule generated by the molecular generation model is evaluated by using Scifinder-n retrosynthetic analysis. Most molecules may be synthesized in two steps. The reason is that the generated molecule is similar to the existing drug sorafenib, ensuring good synthesis. This suggests that a structure-constrained molecule generation model such as a molecular generation model will be an effective tool in a practical work for target-oriented drug discovery. In other words, the in silico analysis result indicates that the sorafenib derivative generated by the molecular generation model may be an alternative drug candidate to sorafenib in patients with high drug resistance.

### CONCLUSION

The AI-based generation model for structure-constrained molecule generation may be not only a solution for effective drug discovery, but also a powerful and explainable tool for chemists and pharmacologists. The existing structure-constrained molecule generation model has limitations in generating molecules that simultaneously satisfy chemical property improvement, novelty, and high similarity to a source molecule. The molecular generation model according to an embodiment of the present disclosure achieves both high property improvement and high structural similarity through two training steps. Besides, it is indicated that the molecular generation model in improving similarity constraints and properties outperforms various state-of-the-art models on four benchmark datasets: DRD2, QED, p log P04, and p log P06.

### Implementation of Detailed Information

The molecular generation model according to an embodiment of the present disclosure is implemented by using several open source tools, including Python 3.6, PyTorch 1.10.1, and RDKit 2021.03.5. RDKit, which is an open source tool for chemical informatics, is used for SMILES kekulization, SMILES validity check, tanimoto similarity calculation, and QED estimation. PyTorch, which is an open source machine learning framework, is used to construct and train the neural network of the molecular generation model. All experiments are performed on Ubuntu 18.04.6 LTS equipped with a memory of 64 GB and GeForce RTX 3090.

### Tanimoto Similarity

Tanimoto similarity, which ranges from 0 to 1, compares molecular structures, which are represented by Morgan fingerprints, such as atomic pairs and topological torsions. In the experimental example of the present disclosure, the Morgan fingerprint is a binary vector generated by using RDKit with a radius of 2 and 2048 bits. With respect to two SMILES strings x and y with corresponding fingerprints “vector FP(x)=(p1, p2, . . . , p2048)” and “FP(y)=(q1, q2, . . . , q2048)”, the tanimoto similarity score is calculated based on Equation 1.

$$𝒯 ( x , y ) = \frac{{\sum }_{i = 1}^{2048} {p}_{i} {q}_{i}}{{\sum }_{j = 1}^{2048} ( {p}_{j} + {q}_{j} - {p}_{j} {q}_{j} )} . \qquad [ Equation 1 ]$$

### Binding Affinity Prediction

Predicting the binding affinity score for ABCG2 and BRAF is very important to apply COMA to sorafenib resistance. In the experimental example of the present disclosure, DeepPurpose, which is a PyTorch-based library for virtual screening, is used for accurate and high throughput affinity prediction for 4.6 million pairs of molecules or more. Furthermore, in BindingDB, which a public database of binding affinity measured to generate training datasets for UGMMT and COMA and to calculate the reward of reinforcement learning in COMA, a prediction model is used for pre-trained message delivery and convolutional neural networks.

### Benchmark Dataset

In this study, four previously provided benchmark datasets shown in Table 1 and an original dataset for sorafenib resistance are used.

TABLE 1

| | | DRD2 | QED | pLogP04 | pLogP06 | Sorafenib |
|---|---|---|---|---|---|---|
| Number | Triplets | 688040 | 1766120 | 1973800 | 1495400 | 4612380 |
| of Unique | (Src, Tar, Neg) | | | | | |
| Items | Pairs (Src, Tar) | 34402 | 88306 | 98690 | 74770 | 230619 |
| | Src | 18490 | 38723 | 57856 | 67718 | 13840 |
| | Tar | 3141 | 13202 | 44759 | 69762 | 2340 |
| | Neg | 21632 | 51923 | 99066 | 132397 | 16180 |
| Range of | (Src, Tar) | 0.40-0.83 | 0.40-0.80 | 0.40-1.00 | 0.60-1.00 | 0.40-1.00 |
| Tanimoto | (Src, Neg) | 0.00-0.30 | 0.00-0.30 | 0.00-0.30 | 0.00-0.49 | 0.03-0.30 |
| Similarity | (Tar, Neg) | 0.00-0.30 | 0.00-0.30 | 0.00-0.30 | 0.00-0.49 | 0.03-0.30 |
| Range of | Src | 0.00-0.05 | 0.70-0.80 | −62.52-1.66 | −32.33-3.89 | 4.90-8.37 |
| Property | Tar | 0.50-1.00 | 0.90-0.95 | −42.76-4.17 | −30.63-5.48 | 3.39-4.70 |
| | Difference | 0.45-1.00 | 0.10-0.25 | 1.00-64.36 | 1.00-23.79 | NA |
| | (Tar − Src) | | | | | |

The DRD2 dataset includes approximately 34,000 molecular pairs (a source and a target) along with the DRD2 activity score derived from the ZINC database. DRD2 activity scores range from 0 to 1 and are evaluated by using a conventional support vector machine regression model. With respect to each pair in the DRD2 dataset, the SMILES string pair satisfies a property constraint that tanimoto similarity is greater than or equal to 0.4. The DRD2 score of the source SMILES string is smaller than 0.05, and the DRD2 score of the target SMILES string is greater than 0.5. The QED dataset included about 88,000 molecular pairs derived from the ZINC database with QED scores. The QED score ranges from 0 to 1 and are calculated by using RDKit. With respect to each pair of QED dataset, tanimoto similarity between two SMILES strings is greater than or equal to 0.4, and QED scores of source and target are in a range of [0.7, 0.8] and [0.9, 1.0], respectively. The penalty log P04 and the penalty log P06 datasets include about 98,000 and about 74,000 molecular pairs derived from the ZINC database together with penalty log P scores. The penalty log P score ranges from −63.0 to 5.5. With respect to each pair of the log P04 dataset to which penalty is applied, the tanimoto similarity between two SMILES strings is greater than or equal to 0.4. In the case of log P06 where a penalty is assigned, the similarity threshold is set to 0.6.

To introduce COMA application cases, a dataset for generating sorafenib-like molecules is constructed. On the basis of the observation that the activity of ABCG2 is associated with sorafenib resistance in hepatocellular carcinoma, this application aims to generate a sorafenib-like molecule with a lower binding affinity for ABCG2 while an affinity level for the target kinase BRAF is preserved. This dataset includes about 230,000 molecular pairs derived from the ChEMBL database together with binding affinity scores for ABCG2 and BRAF. The binding affinity score evaluated by using DeepPurpose is pKd. With respect to each pair in the ABCG2 dataset, the tanimoto similarity between two molecules is greater than or equal to 0.4, and the ABCG2 affinity values of a source and a target are in the range of [4.9, 8.4] and [3.3, 4.7], respectively. In the case of BRAF, binding affinity for each of the source and the target is greater than 6.0.

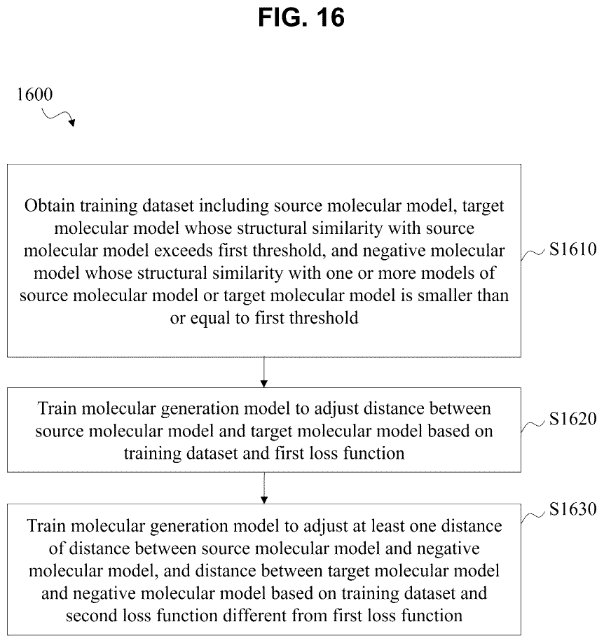

FIG. 16 is a flowchart of a training method 1600 for a molecular generation model, according to an embodiment of the present disclosure. A method 1600 may be performed by at least one processor (e.g., the processor 414) of a computing device. In the meantime, as illustrated, the method 1600 may start with step S1610 of obtaining a training dataset including a source molecular model, a target molecular model whose structural similarity with the source molecular model exceeds a first threshold, and a negative molecular model whose structural similarity with one or more models of the source molecular model or the target molecular model is smaller than or equal to the first threshold.

The processor may train a molecular generation model to adjust a distance between the source molecular model and the target molecular model based on the training dataset and a first loss function (S1620). For example, the processor may train the molecular generation model to decrease the distance between the source molecular model and the target molecular model based on the training dataset and the first loss function.

The processor may train the molecular generation model to adjust at least one distance of a distance between the source molecular model and the negative molecular model, and a distance between the target molecular model and the negative molecular model based on the training dataset and a second loss function different from the first loss function (S1630). The processor may train the molecular generation model to increase at least one distance of the distance between the source molecular model and the negative molecular model, and the distance between the target molecular model and the negative molecular model, based on the training dataset and the second loss function different from the first loss function.

Additionally, the processor may train the molecular generation model such that a molecular model whose structural similarity with the source molecular model exceeds a second threshold is output from the source molecular model, based on the training dataset and a reward function. For example, the processor may obtain an output molecular model by entering the source molecular model into the molecular generation model, and calculating a positive weight or a negative weight associated with the output molecular model and may assign the positive weight or the negative weight to the molecular generation model, based on whether structural similarity between the output molecular model and the source molecular model exceeds the second threshold as a result of comparing the output molecular model and the source molecular model. For another example, the processor may calculate the positive weight or the negative weight associated with the output molecular model and the assigning the positive weight or the negative weight to the molecular generation model, based on whether the structural similarity between the output molecular model and the source molecular model exceeds the second threshold, and whether a chemical property score of the output molecular model exceeds a chemical property score of the source molecular model as the results of comparing the output molecular model and the source molecular model. For still another example, the processor may train the molecular generation model such that a molecular model whose structural similarity with the source molecular model exceeds the second threshold and which has a chemical property score greater than the chemical property score of the source molecular model, is output from the source molecular model, based on the training dataset and the reward function.

In the meantime, the molecular generation model may be configured such that a chemical property score of the target molecular model may be greater than a chemical property score of the source molecular model.

Various modifications of the present disclosure will be easily apparent to those skilled in the art, and the generic principles defined herein may be applied to various modifications without departing from the spirit or scope of the present disclosure. Accordingly, the present disclosure is not intended to be limited to the examples set forth herein, but is to be accorded the widest scope consistent with the principles and novel features disclosed herein.

Although example implementations may refer to utilizing aspects of the presently disclosed subject matter in the context of one or more standalone computer systems. However, the subject matter is not so limited, but rather may be implemented in connection with any computing environment, such as a network or a distributed computing environment. Furthermore, the aspects of the presently disclosed subject matter may be implemented in or across a plurality of processing chips or devices, and the storage may be similarly affected across a plurality of devices. These devices may include PCs, network servers, and handheld devices.

Although the present disclosure has been described herein in connection with some embodiments, it should be understood that various modifications and changes may be made without departing from the scope of the present disclosure as understood by those skilled in the art to which the present disclosure pertains. Moreover, such modifications and variations are intended to fall within the scope of claims appended hereto.

1. A training method for a molecular generation model performed by at least one processor, the method comprising:
obtaining a training dataset including a source molecular model, a target molecular model whose structural similarity with the source molecular model exceeds a first threshold, and a negative molecular model whose structural similarity with one or more models of the source molecular model or the target molecular model is smaller than or equal to the first threshold;
training a molecular generation model to adjust a distance between the source molecular model and the target molecular model based on the training dataset and a first loss function; and
training the molecular generation model to adjust at least one distance of a distance between the source molecular model and the negative molecular model, and a distance between the target molecular model and the negative molecular model based on the training dataset and a second loss function different from the first loss function.

2. The method of claim 1, wherein the training of the molecular generation model to adjust the distance between the source molecular model and the target molecular model based on the training dataset and the first loss function includes:
training the molecular generation model to decrease the distance between the source molecular model and the target molecular model based on the training dataset and the first loss function.

3. The method of claim 1, wherein the training of the molecular generation model to adjust the at least one distance of the distance between the source molecular model and the negative molecular model, and the distance between the target molecular model and the negative molecular model based on the training dataset and the second loss function different from the first loss function includes:
training the molecular generation model to increase the at least one distance of the distance between the source molecular model and the negative molecular model, and the distance between the target molecular model and the negative molecular model, based on the training dataset and the second loss function different from the first loss function.

4. The method of claim 1, further comprising:
training the molecular generation model such that a molecular model whose structural similarity with the source molecular model exceeds a second threshold is output from the source molecular model, based on the training dataset and a reward function.

5. The method of claim 4, wherein the training of the molecular generation model such that the molecular model whose structural similarity with the source molecular model exceeds the second threshold is output from the source molecular model, based on the training dataset and the reward function includes:
obtaining an output molecular model by entering the source molecular model into the molecular generation model; and
calculating a positive weight or a negative weight associated with the output molecular model and assigning the positive weight or the negative weight to the molecular generation model, based on whether structural similarity between the output molecular model and the source molecular model exceeds the second threshold as a result of comparing the output molecular model and the source molecular model.

6. The method of claim 5, wherein the calculating of the positive weight or the negative weight associated with the output molecular model and the assigning of the positive weight or the negative weight to the molecular generation model, based on whether the structural similarity between the output molecular model and the source molecular model exceeds the second threshold as the result of comparing the output molecular model and the source molecular model includes:
calculating the positive weight or the negative weight associated with the output molecular model and the assigning the positive weight or the negative weight to the molecular generation model, based on whether the structural similarity between the output molecular model and the source molecular model exceeds the second threshold, and whether a chemical property score of the output molecular model exceeds a chemical property score of the source molecular model as the results of comparing the output molecular model and the source molecular model.

7. The method of claim 4, wherein the training of the molecular generation model such that the molecular model whose structural similarity with the source molecular model exceeds the second threshold is output from the source molecular model, based on the training dataset and the reward function includes:
training the molecular generation model such that a molecular model whose structural similarity with the source molecular model exceeds the second threshold and which has a chemical property score greater than a chemical property score of the source molecular model, is output from the source molecular model, based on the training dataset and the reward function.

8. The method of claim 1, wherein a chemical property score of the target molecular model is greater than a chemical property score of the source molecular model.

9. A computer-readable recording medium which records a computer program to perform the training method for a molecular generation model according to claim 1.

10. A training device for a molecular generation model, the training device comprising:
a memory configured to store data associated with the molecular generation model; and
at least one processor connected to the memory and configured to train the molecular generation model,
wherein the at least one processor includes instructions, the instructions, when executed by the at least one processor, causing the at least one processor to:
obtain a training dataset including a source molecular model, a target molecular model whose structural similarity with the source molecular model exceeds a first threshold, and a negative molecular model whose structural similarity with one or more models of the source molecular model or the target molecular model is smaller than or equal to the first threshold;
train the molecular generation model to adjust a distance between the source molecular model and the target molecular model based on the training dataset and a first loss function; and
train the molecular generation model to adjust at least one distance of a distance between the source molecular model and the negative molecular model, and a distance between the target molecular model and the negative molecular model based on the training dataset and a second loss function different from the first loss function.

11-17. (canceled)
